(19) United States
(12) Plant Patent Application Publication
(10) Pub. No.: US 2002/0188976 P1
(43) Pub. Date: Dec. 12, 2002

(54) Chrysanthemum plant named 'Moza Can 01'

(76) Inventor: Frank Moser, Alva, FL (US)

Correspondence Address: KNOBBE MARTENS OLSON & BEAR LLP, 2040 MAIN STREET, FOURTEENTH FLOOR, IRVINE, CA, 92614, US

(21) Appl. No.: 09/865,149

(22) Filed: May 24, 2001

(51) Int. Cl. A01H 5/00

(52) U.S. Cl. PLT/287

(57) ABSTRACT

Abstract of the Disclosure A new and distinct Chrysanthemum plant variety characterized by a daisy type inflorescence with pink/white color ray florets, flexible plant habit, vigorous growth habit, and consistent natural season flowering habit during late September.

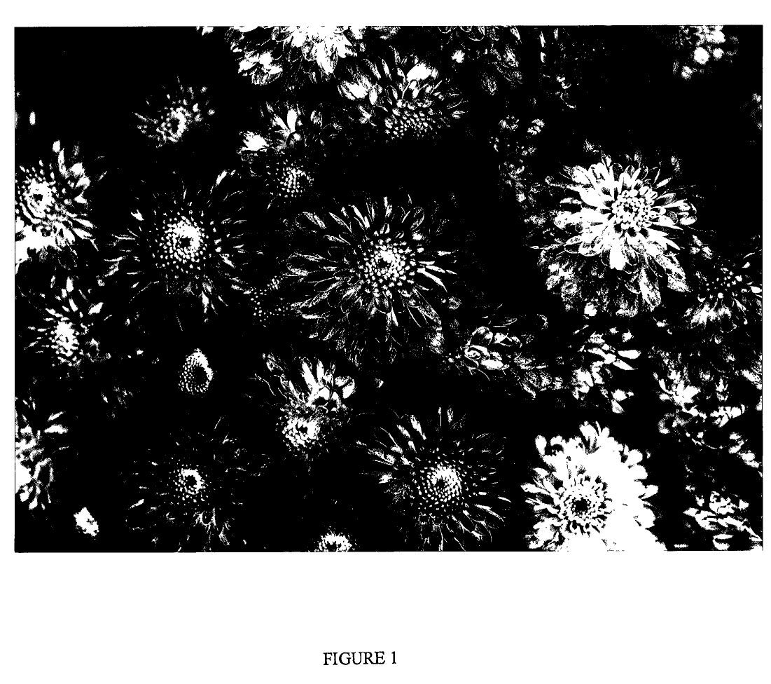

### BACKGROUND OF THE INVENTION

The present invention relates to a new and distinct cultivar of Chrysanthemum plant, botanically known as Dendranthema morifolium and herein after referred to by the cultivar name ‘MOZA CAN 01.’

This new cultivar originated from hybridization of a female parent Chrysanthemum cultivar (unpatented in the United States) with a male parent Chrysanthemum cultivar (unpatented in the United States). The new cultivar was discovered by the inventor in November 1998.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘MOZA CAN 01’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘MOZA CAN 01.’ These characteristics in combination distinguish ‘MOZA CAN 01’ as a new and distinct Chrysanthemum:

1. Small, decorative type inflorescence;

2. Unique pink/yellow ray floret coloration;

3. Small foliage;

4. Compact growth habit; and

5. Consistent natural season flowering habit during late September.

Plants of the new cultivar are similar to plants of the female parent cultivar in most horticultural characteristics, however plants of the new cultivar differed from plants of the female parent cultivar in the following characteristics:

1. The new cultivar has different color ray florets;

2. The new cultivar is shorter;

3. The new cultivar has a different type bloom; and

4. The new cultivar has shorter internode length.

Plants of the new cultivar are similar to plants of the male parent cultivar in most horticultural characteristics, however plants of the new cultivar differed from plants of the male parent cultivar in the following characteristics:

1. The new cultivar has different color ray florets;

2. The new cultivar has a smaller diameter bloom; and

3. The new cultivar has shorter internode length.

### BRIEF DESCRIPTION OF THE PHOTOGRAPHS

The photograph shows a close up view of the flowers and buds of the new cultivar.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘MOZA CAN 01’

Commercial classification: Garden-type Chrysanthemum

Parentage: Hybridization of female parent Chrysanthemum cultivar (unpatented in the United States) with a male parent Chrysanthemum cultivar (unpatented in the United States)

Propagation

Time to rooting.—7 to 14 days at approximately 21° C.

Rooting habit.—Fine, fibrous

Plant

Growth habit.—Mounding herbaceous perennial

Height.—Approximately 22 cm

Growth rate.—Moderate

Branching characteristics.—Free Branching

Length of lateral branches.—Approximately 18 cm

Number of leaves per lateral branch.—Approximately 15

Stem color.—Near 138B

Foliage

Leaf.—Arrangement: Alternate single Average Length: Approximately 5.0 cm Average Width: Approximately 3.0 cm Shape of blade: Ovate Apex: Acuminate Base: Attenuate Attachment: Stalked Margin: Serrate Texture of top surface: Lightly pubescent Texture of bottom surface: Lightly pubescent Color: young foliage upper side: Near 138A young foliage under side: Near 138A mature foliage upper side: Near 138A mature foliage underside: Near 138A venation upper side: Near 138B venation under side: Near 137B

Petiole.—Average Length: Approximately 1.6 cm

Bloom

Inflorescence.—Flowering habit: Induced by darkness period greater than 13.5 hours, approximately 49 days of appropriate day length required to induce and develop blooms Inflorescence form: Decorative Natural flowering season: Approximately the last two weeks in September Number of inflorescences per stem: Approximately 16 Inflorescence diameter: Approximately 3.5 cm Inflorescence depth: Approximately 2.5 cm Inflorescence longevity on plant: Approximately 22 to 26 days Persistence: Persistent

Ray Florets.—Appearance: Matte Texture: Smooth Average Number per flower: Approximately 200 Shape: Oblanceolate Margin: Entire Apex: Acute Length: Approximately 1.8 cm Width: Approximately 0.5 cm Color: upper surface at first opening: Near 39A at the tip; Near 15C at the base upper surface at maturity: Near 46D at the tip; Near 40D at the base upper surface at fading: Near 37A at the tip; Near 24D at the base under surface at first opening: Near 13A under surface at maturity: Near 33C at the tip; Near 16D at the base under surface at fading: Near 33D at the tip; Near 20C at the base

Disc florets.—Appearance: Shiny Texture: Smooth Average number perflower: Approximately 20 Shape: Cylindric Apex: Obtuse Average Length: Approximately 0.5 cm Average Width: Approximately 0.1 cm Color: at first opening: Near 154B at maturity: Near 4A at fading: Near 7A

Peduncle.—Length: at terminal end (shortest): Approximately 4 cm at lateral end (longest): Approximately 5.5 cm Strength: Moderate Color: Near 138A Habit: Erect

Infloreseence bud.—Length: Approximately 0.9 cm Diameter: Approximately 0.9 cm Form: Globular Color: Near 42B

Sepals.—Appearance: Matte Texture: Lightly pubescent Number: Approximately 20 to 30 Shape: Ovate Margin: Entire Apex: Acute Color: upper side: Near 137A underside: Near 137A

Reproductive Organs

Ray Florets.—Pistillate: Number ofpistils per flower: 1 Stigma shape: 2 branched Stigma color: Near 4A Style color: Near 2D Style length: Approximately 0.5 cm

Disc Florets.—Pistillate: Number of pistils perflower: 1 Stigma shape: Cylindric Style length: Approximately 0.3 cm Staminate: Number ofstamens per flower: Approximately 5 Anther shape: Tubular Anther color: Near 5B Pollen color: Near 5A

Other Characteristics

Disease Resistance.—Neither resistance nor susceptibility observed

‘MOZA CAN 01’: Table of R.H.S. Color Codes

| | | | |
|---|---|---|---|
| FOLIAGE | Leaf | young foliage upper surface | Near 138A |
| | | young foliage under surface | Near 138A |
| | | mature foliage upper surface | Near 138A |
| | | mature foliage under surface | Near 138A |
| | | venation upper surface | Near 138B |
| | | venation under surface | Near 137B |
| BLOOM | Ray Florets | upper surface at first opening, | Near 39A |
| | | tip | |
| | | upper surface at first opening, | Near 15C |
| | | base | |
| | | upper surface at maturity, tip | Near 46D |
| | | upper surface at maturity, base | Near 40D |
| | | upper surface at fading, tip | Near 37A |
| | | upper surface at fading, base | Near 24D |
| | | under surface at first opening | Near 13C |
| | | under surface at maturity, tip | Near 33C |
| | | under surface at maturity, base | Near 16D |
| | | under surface at fading, tip | Near 33D |
| | | under surface at fading, base | Near 20C |
| | | Pistillate: Stigma | Near 4A |
| | | Pistillate: Style | Near 2D |
| | Disc Florets | at first opening | Near 154B |
| | | at maturity | Near 4A |
| | | at fading | Near 7A |
| | | Staminate: Anther | Near 5B |
| | | Staminate: Pollen | Near 5A |
| | Peduncle | | Near 138A |
| | Inflorescence | | Near 42B |
| | Bud | | |
| | Sepals | upper surface | Near 137A |
| | | under surface | Near 137A |

What is claimed is:

1. A new and distinct variety of Chrysanthemum plant named ‘MOZA CAN 01’ as herein illustrated and described.
